(19) United States
(12) Patent Application Publication
(10) Pub. No.: US 2023/0274659 A1
(43) Pub. Date: Aug. 31, 2023

(54) SYSTEMS AND METHODS FOR PROVIDING GUIDED DIALYSIS TRAINING AND SUPERVISION

(71) Applicant: Chudi Adi, Albuquerque, NM (US)

(72) Inventor: Chudi Adi, Albuquerque, NM (US)

(21) Appl. No.: 18/113,820

(22) Filed: Feb. 24, 2023

Related U.S. Application Data
(60) Provisional application No. 63/317,479, filed on Mar. 7, 2022.
(60) Provisional application No. 63/314,285, filed on Feb. 25, 2022.

(51) Int. Cl. G09B 19/00; A61M 1/14; G09B 5/02; G06T 19/00; G06V 20/20; G06V 20/52; G06T 7/70; G06T 7/00; G06V 40/10

(52) U.S. Cl. CPC G09B 19/003; A61M 1/14; G06T 7/0012; G06T 7/70; G06T 19/006; G06V 20/20; G06V 20/52; G06V 40/10; G09B 5/02; A61M 2205/52; G06T 2207/30004; G06T 2207/30196; G06V 2201/034

(57) ABSTRACT

A dialysis path includes dialysis steps such as a machine interaction step. A machine state input receives dialysis machine status information for a dialysis machine. An instruction output provides instructional information for a dialysis procedure for a patient. A dialysis process state is used to identify completion of the dialysis steps. A user performing the dialysis steps in a proper order causes the dialysis process state to traverse the dialysis path of a dialysis procedure, beginning at a first dialysis step and ending at a last dialysis step. The machine interaction step includes a user interaction with the dialysis machine that causes the dialysis machine status information, changes the dialysis process state, and completes the machine interaction step. Current step information in the instructional information guides the user to completing a current step. The instruction output provides the current step information to the user.

### CROSS REFERENCE TO RELATED APPLICATIONS

This patent application claims the priority and benefit of U.S. provisional patent application no. 63/314,285, titled “The RenaVis Telehealth and Telemonitoring system,” filed on Feb. 25, 2022 and also claims the priority and benefit of U.S. provisional patent application no. 63/317,479, titled “XRASP Stethoscope System,” filed on Mar. 7, 2022. U.S. provisional patent application no. 63/314,285 and U.S. provisional patent application no. 63/317,479 are herein incorporated by reference in their entirety.

### TECHNICAL FIELD

The embodiments relate to dialysis, home dialysis, training for home dialysis, and to using virtual reality and augmented reality capabilities to train users to perform dialysis at home.

### BACKGROUND

Patients requiring dialysis often go to dialysis centers where a dialysis procedure is performed on the patient. The patients may require weekly or more often dialysis procedures. The costs of performing dialysis procedures at the patient’s home are much less than the costs of using dialysis centers and the outcomes are often better because the patient is not transported or exposed to the hospital setting.

### BRIEF SUMMARY OF SOME EXAMPLES

The following presents a summary of one or more aspects of the present disclosure, in order to provide a basic understanding of such aspects. This summary is not an extensive overview of all contemplated features of the disclosure and is intended neither to identify key or critical elements of all aspects of the disclosure nor to delineate the scope of any or all aspects of the disclosure. Its sole purpose is to present some concepts of one or more aspects of the disclosure in a form as a prelude to the more detailed description that is presented later.

One aspect of the subject matter described in this disclosure can be implemented by a system. The system can include a memory that stores a dialysis process state and a dialysis path that includes a plurality of dialysis steps that includes a machine interaction step, a machine state input that receives dialysis machine status information for a dialysis machine, an instruction output that provides instructional information for a dialysis procedure for a patient, and a processor that uses the dialysis process state to identify completion of the dialysis steps, wherein a user performing the dialysis steps in a proper order causes the dialysis process state to traverse the dialysis path from a first dialysis step to a last dialysis step, the dialysis procedure begins at the first dialysis step and completes at the last dialysis step, the machine interaction step includes a user interaction with the dialysis machine that causes the dialysis machine status information to change the dialysis process state to thereby complete the machine interaction step, the instructional information includes a current step information that guides the user to completing a current step, and the instruction output provides the current step information to the user.

Another aspect of the subject matter described in this disclosure can be implemented by a method. The method can include storing a dialysis process state in a memory, and storing, in the memory, a dialysis path that includes a plurality of dialysis steps that includes a machine interaction step. The method may also include receiving a dialysis machine status information for a dialysis machine, providing, to a user, instructional information for a dialysis procedure for a patient, and using the dialysis process state to identify completion of the dialysis steps, wherein the user performing the dialysis steps in a proper order causes the dialysis process state to traverse the dialysis path from a first dialysis step to a last dialysis step, the dialysis procedure begins at the first dialysis step and completes at the last dialysis step, the machine interaction step includes a user interaction with the dialysis machine that produces dialysis machine status information that changes the dialysis process state to thereby complete the machine interaction step, and the instructional information includes a current step information that guides the user to completing a current step.

Yet another aspect of the subject matter described in this disclosure can be implemented by a system. The system can include a means for storing a dialysis process state and a dialysis path that includes a plurality of dialysis steps that includes a step for machine interaction, a means for using a dialysis machine status information for a dialysis machine to change the dialysis process state, an instructive means for instructing a user for performing a dialysis procedure for a patient, and a means identifying completion of the dialysis steps using dialysis process state, wherein the user performing the dialysis steps in a proper order causes the dialysis process state to traverse the dialysis path from a first dialysis step to a last dialysis step, the dialysis procedure begins at the first dialysis step and completes at the last dialysis step, the step for machine interaction produces dialysis machine status information that changes the dialysis process state to thereby complete the step for machine interaction, and the instructive means includes a means for guiding the user to complete a current step.

In some implementations of the methods and devices, the system further includes an imaging input, wherein the dialysis machine is a physical dialysis machine, the imaging input receives a sequence of images of a control panel of the dialysis machine, and the dialysis machine status information is determined using the images of the control panel. In some implementations of the methods and devices, a user training state tracks a training level of the user, the user training state is used to determine the instructional information that is presented to the user, and the user training state is used to select a hint trigger that triggers display of the instructional information to the user. In some implementations of the methods and devices, the dialysis machine is a virtual dialysis machine, and the user interacts with the virtual dialysis machine to thereby change the dialysis machine status information. In some implementations of the methods and devices, a 3D model of the dialysis machine is used to present the dialysis machine to the user in augmented reality, mixed reality, or extended reality.

In some implementations of the methods and devices, the instructional information is presented to the user in augmented reality, mixed reality, or extended reality, the dialysis machine is a physical dialysis machine, and a current dialysis step is used to determine a hint location at which the instructional information appears to the user. In some implementations of the methods and devices, the system further includes an imaging input that receives a plurality of images, and an object recognizer that recognizes a dialysis supply item in the images, wherein the dialysis steps include a supply confirmation step, the dialysis supply item is imaged in the images, the object recognizer uses the images to confirm that the dialysis supply item is present, and the supply confirmation step is completed by confirming that the dialysis supply item is present. In some implementations of the methods and devices, the system further includes an imaging input that receives a plurality of images, and an object recognizer that recognizes a plurality of dialysis supply items in the images, wherein the dialysis supply items include a clamp, a tube, and a dialysis bag, the dialysis steps include a supply confirmation step, the dialysis supply items are imaged in the images, the object recognizer uses the images to confirm that the dialysis supply items are present, and the supply confirmation step is completed by confirming that the dialysis supply items are present.

In some implementations of the methods and devices, the system further includes an imaging input that receives a plurality of images, and an object recognizer that recognizes a first dialysis supply item and a second dialysis supply item, wherein the dialysis steps include an item positioning step that includes confirming that the first dialysis supply item is properly positioned relative the second dialysis supply item, the first dialysis supply item and the second dialysis supply item are imaged in the images, the object recognizer uses the images to determine a first item position of the first dialysis supply item and a second item position of the second dialysis supply item, and the item positioning step is completed by determining that the first item position relative to the second item position meets a positioning criterion. In some implementations of the methods and devices, the first dialysis supply item is a tube and the second dialysis supply item is a clamp.

In some implementations of the methods and devices, the system further includes an imaging input that receives a plurality of images, and an object recognizer that recognizes a body part of the patient and a dialysis supply item, wherein, the dialysis steps include a body contact step that includes confirming that the dialysis supply item is properly positioned relative the body part, the body part and the dialysis supply item are imaged in the images, the object recognizer uses the images to determine an item position of the dialysis supply item and a body part position of the body part, and the body contact step is completed by determining that the item position relative to the body part position meets a positioning criterion. In some implementations of the methods and devices, the dialysis supply item is a dialysis needle.

In some implementations of the methods and devices, the current step information is provided to the user as an overlay that appears over the dialysis machine, and the dialysis machine is a physical dialysis machine. In some implementations of the methods and devices, the current step information is provided to the user by a virtual avatar that interacts with a virtual dialysis machine or virtual dialysis supply items. In some implementations of the methods and devices, the method further includes receiving a sequence of images of a control panel of the dialysis machine, and using the images of the control panel to determine the dialysis machine status information, wherein the dialysis machine is a physical dialysis machine.

These and other aspects will become more fully understood upon a review of the detailed description, which follows. Other aspects, features, and embodiments will become apparent to those of ordinary skill in the art, upon reviewing the following description of specific, exemplary embodiments in conjunction with the accompanying figures. While features may be discussed relative to certain embodiments and figures below, all embodiments can include one or more of the advantageous features discussed herein. In other words, while one or more embodiments may be discussed as having certain advantageous features, one or more of such features may also be used in accordance with the various embodiments discussed herein. In similar fashion, while exemplary embodiments may be discussed below as device, system, or method embodiments such exemplary embodiments can be implemented in various devices, systems, and methods.

### BRIEF DESCRIPTION OF THE DRAWINGS

FIG. 1 is a high-level conceptual diagram of a virtual avatar guiding a user, who is also the patient, through a virtual dialysis procedure according to some aspects.

FIG. 2 is a high-level block diagram of a host machine that can provide guided dialysis training and supervision, according to some embodiments.

FIG. 3 is a high-level block diagram of a software system that can provide guided dialysis training and supervision, according to some embodiments.

FIG. 4 is a high-level conceptual diagram of a supply confirmation step being completed, according to aspects of the embodiments.

FIG. 5A is a high-level conceptual diagram of an item positioning step being completed, according to aspects of the embodiments.

FIG. 5B is a high-level conceptual diagram of a body contact step being completed, according to aspects of the embodiments.

FIG. 6 is a high-level conceptual diagram of a machine interaction step being completed, according to aspects of the embodiments.

FIG. 7 is a high-level conceptual diagram of a process coordinator using a dialysis path to guide a user through the proper order of dialysis steps of a dialysis procedure, according to aspects of the embodiments.

FIG. 8 is a high-level conceptual diagram of a process coordinator using a user training state to guide selection of a physical step or a mixed reality step as the current dialysis step, aspects of the embodiments.

FIG. 9 is a high-level conceptual diagram of a mixed reality dialysis step being completed, according to aspects of the embodiments.

FIG. 10 is a high-level flow diagram of a process that a process coordinator may use to guide a user through a dialysis procedure, according to aspects of the embodiments.

FIG. 11 is a high-level conceptual diagram of current step information being presented to a user, according to aspects of the embodiments.

FIG. 12 is a high-level flow diagram of using a user training state to adjust the training of the user, according to aspects of the embodiments.

FIG. 13 is a high-level block diagram of a software system that can use a virtual avatar to provide guided dialysis training and supervision, according to some embodiments.

FIG. 14 is a high-level flow diagram illustrating a method for providing guided dialysis training and supervision, according to some embodiments.

FIG. 15 is a high-level conceptual diagram of a virtualized avatar guiding a patient that is using a remotely readable stethoscope, according to aspects of the embodiments.

Throughout the description, similar reference numbers may be used to identify similar elements.

### DETAILED DESCRIPTION

It will be readily understood that the components of the embodiments as generally described herein and illustrated in the appended figures could be arranged and designed in a wide variety of different configurations. Thus, the following more detailed description of various embodiments, as represented in the figures, is not intended to limit the scope of the present disclosure, but is merely representative of various embodiments. While the various aspects of the embodiments are presented in drawings, the drawings are not necessarily drawn to scale unless specifically indicated.

The present invention may be embodied in other specific forms without departing from its spirit or essential characteristics. The described embodiments are to be considered in all respects only as illustrative and not restrictive. The scope of the invention is, therefore, indicated by the appended claims rather than by this detailed description. All changes which come within the meaning and range of equivalency of the claims are to be embraced within their scope.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussions of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain embodiments that may not be present in all embodiments of the invention.

Reference throughout this specification to “one embodiment”, “an embodiment”, or similar language means that a particular feature, structure, or characteristic described in connection with the indicated embodiment is included in at least one embodiment of the present invention. Thus, the phrases “in one embodiment”, “in an embodiment”, and similar language throughout this specification may, but do not necessarily, all refer to the same embodiment.

For patients requiring regular and scheduled dialysis, dialysis procedures that are performed in a home setting are more cost effective than dialysis procedures performed at a hospital or dialysis center. Furthermore, the patient outcomes are often better because the patient is not exposed to the stress of transport and treatment away from home. In addition, patients may be exposed to diseases during transport, at a hospital, or at a dialysis center. The difficulty is in training the patient, a caretaker, or both to perform dialysis procedures at home. The training may take many sessions as the trainee becomes accustomed to the idea of performing a medical procedure and becomes familiar with the dialysis machine and the supplies that are needed for performing the procedure. A further aspect is that a patient or caretaker performing a home dialysis procedure may want the attention of a healthcare professional during the procedure or something may happen that indicates that a healthcare professional should check in on the procedure.

Advances in augmented reality and virtual reality are providing opportunities for training people to perform numerous tasks. For example, hardware and software systems are currently available that can perform full body tracking of a person, that can recognize, locate, and analyze physical objects in images or sequences of images (e.g., video), etc. Many of these same systems can place interactive and noninteractive virtual objects in a user’s virtual environment or augmented environment. Interactive virtual objects are objects that the user can interact with by moving the object, operating virtualized equipment, etc. Here, virtual reality refers to providing a user with a completely virtual environment to interact with. Augmented reality refers to providing the user with an augmented environment that is an augmented version of the physical environment. The augmented environment can include virtual objects within the user’s augmented environment such that the user can see or interact with the virtual objects. The virtual objects can include virtualized dialysis machines and virtualized dialysis supplies such as dialysis bags, clamps, tubes, dialysis needles, and dialysis cartridges. The augmented environment can also include information that appears to overlay or be near a physical object or virtual object to thereby provide information related to that object. For example, the instruction can instruct the user to place a dialysis bag, which may contain fluids for use in the dialysis procedure, into a heater. Another instruction can instruct the user to turn on the heater. Yet another instruction can instruct the user to wait until the heater indicates the bag is warmed to an acceptable temperature (e.g., greater than a lower threshold, within a temperature range, etc.). In this manner, an entire dialysis procedure may be broken down into steps for the user to perform and each of those steps can include instructions and include conditions that must be met in order for the step to be complete.

For physical dialysis equipment and machines, images of the patient setting can include images of the patient, of the dialysis machine, of the dialysis supplies, etc. The images may be analyzed to locate the dialysis machine, to locate the control panel of the dialysis machine, and to determine the status of the dialysis machine (e.g., on/off, initialized, ready to operate, operating, fluid flow measurements, etc.). Software is commercially and freely available that is capable of the image analysis required for determining the status and presence and location of the patient, the dialysis machine, and the dialysis supplies. Software and hardware solutions are available that can perform full body tracking of the patient. These software and hardware solutions may be used to determine the location, position, and status of objects and people. That physical location, position, and status information may be compared to desired location, position, and status information to determine if a dialysis step is complete. Once one step is complete, similar operations may be performed to determine when the next step is complete.

For virtual dialysis equipment, the training system can place the dialysis machine and dialysis supplies in the user’s augmented environment. As such, the positions, locations, and statuses of the dialysis machine and dialysis supplies are known and do not have to be determined. The patient’s location, position, and status can be determined, as discussed above, through the analysis of images or via any of the commercially or freely available body tracking systems. The user may interact with the virtual objects similarly to how users interact with virtual objects in various well-known virtual or augmented environments.

A user can be trained by running the user through a series of training scenarios. Each training scenario can be defined by a dialysis path. A dialysis path can be a sequence of steps that the user is to follow in a proper order. An example of a proper order is proceeding from a first step to a last step in a path. In the earlier training stages, the dialysis path may consist entirely of steps that involve virtual dialysis machines and virtual dialysis supplies. In those early training stages, the user may be provided with instructions immediately at the start of the step. In later training stages, the instructions may be delayed such that the user may complete the step without receiving the instruction. Furthermore, more advanced training stages may use physical dialysis machines and physical dialysis supplies. A physical step may be interrupted such that the user may perform a virtual version of the step as a form of instruction and then returned to the physical step. A coach or monitor may monitor the user’s progress through the dialysis path and may provide additional guidance through voice, text, video, or virtual avatar. In some cases, the system may notice a problem. For example, the camera may recognize bleeding or leaking of fluid. In such cases the system may alert the user and the coach/monitor in order to address the problem.

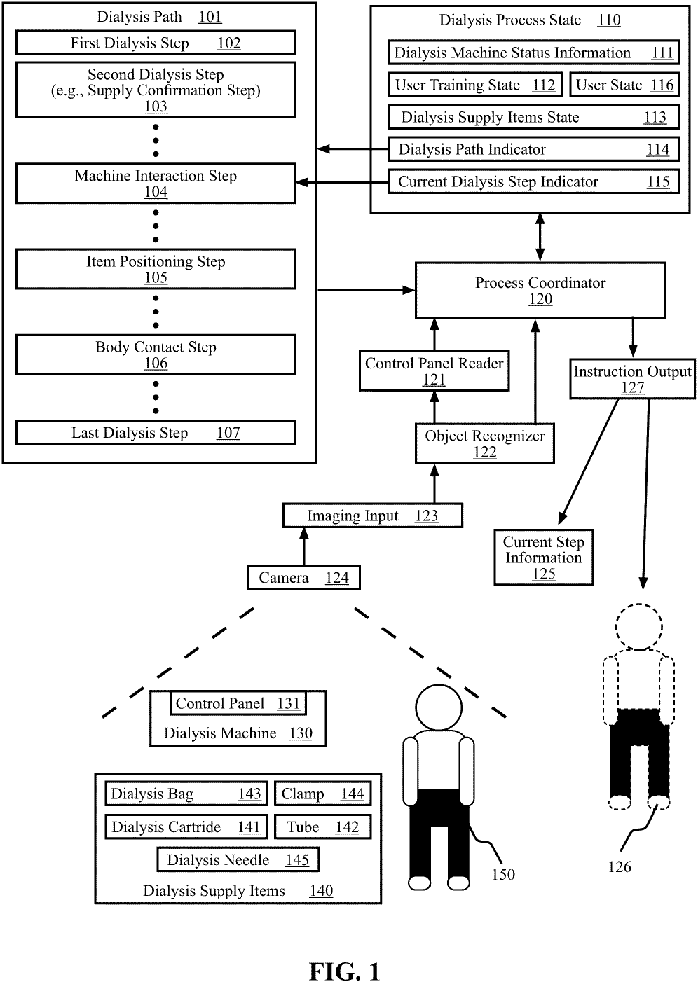

FIG. 1 is a high-level conceptual diagram of a virtual avatar 126 guiding a user 150, who is also the patient, through a simulated dialysis procedure according to some aspects. The user 150 has a dialysis machine 130 and dialysis supply items 140. The dialysis machine has a control panel 131 that may include switches, digital readouts (e.g., numerical or alphanumeric text), analog readouts (e.g., dials), and indicator lights. An indicator light can change color, turn on, or turn off to indicate a machine status. The dialysis supply items 140 can include a dialysis bag 143, a clamp 144, a dialysis cartridge 141, a tube 142, a dialysis needle 145, and other items. A camera 124 can image the user 150, the dialysis machine 130, and the dialysis supply items 140. The camera 124 can provide a sequence of images to an imaging input 123 that receives the images and provides the images to an object recognizer 122. An image produced by the camera 124 may include an image of the control panel 131, an image of the dialysis machine 130, images of the dialysis supply items 140, and an image of the user 150. Current commercially and freely available image recognizers are already trained to recognize people and some objects. Such recognizers are typically designed such that they can be easily trained to recognize additional objects. As such, the object recognizer 122 may produce data that indicates the locations and orientations of the user 150, dialysis machine 130, and the dialysis supply items 140. Furthermore, the data produced by the object recognizer may indicate the locations and orientations of the user’s body parts such as the user’s hands, arms, legs, and torso.

The images produced by the camera may include images of the control panel 131 of the dialysis machine 130. A control panel reader 121 can use images of the control panel 131 to determine the status of the dialysis machine 130. The control panel reader 121 can produce dialysis machine status information 111 for the dialysis machine 130 that indicates the state of the dialysis machine 130. The dialysis machine status information 111 may also include the location and orientation of the dialysis machine 130. A process coordinator 120 can receive the dialysis machine status information 111 and can also receive the locations and orientations of the user 150, dialysis machine 130, and the dialysis supply items 140. The dialysis machine status information 111 can be stored as part of a dialysis process state 110. The dialysis process state may also include a user training state 112, a dialysis supply items state 113, a dialysis path indicator 114, a current dialysis step indicator 115, and a user state 116. The user state 116 can indicate the locations and orientations of the user 150 and the user’s body parts. The dialysis supply items state 113 can indicate the locations and orientations of the dialysis supply items 140. The dialysis path indicator 114 can indicate the dialysis path 101 that the user 150 follows to perform the dialysis procedure. The current dialysis step indicator 115 can indicate which step of the dialysis procedure is currently being performed. The user training state 112 can indicate a training level for the user 150 and may be used to select a dialysis path 101.

The dialysis path 101 can include the dialysis steps that are to be performed. The dialysis steps can be ordered in a proper order such that a first dialysis step 102 is to be performed first and before a second dialysis step 103 and so forth until a last dialysis step 107 is performed. The user can perform a dialysis procedure by performing the dialysis steps in the proper order. Performing a dialysis step causes the dialysis process state 110 to change and performing the dialysis steps in the proper order causes the dialysis process state to traverse the dialysis path from the first dialysis step 102 to the last dialysis step 107. The dialysis steps in the dialysis path 101 can include a machine interaction step 104, an item positioning step 105, and a body contact step 106. In a machine interaction step, the user interacts with the dialysis machine and causes the dialysis machine status information 111 to change. The dialysis process state 110 changes when the dialysis machine status information 111 changes, the user training state 112 changes, the dialysis supply items state 113 changes, the dialysis path indicator 114 changes, the current dialysis step indicator 115 changes, or the user state 116 changes.

A dialysis step can include step information that can be presented to the user in order to guide the user toward completing the dialysis step. Current step information 125 from the current dialysis step, which is indicated by the current dialysis step indicator 115, can be presented to the user by an instruction output 127. The instruction output 127 may produce a virtual avatar 126 within the user’s augmented environment or virtual environment. The virtual avatar may say the current step information, read the current step information aloud, etc. The current step information 125 may be positioned such that it overlays the control panel 131, the dialysis machine 130, a body part of the user, or any of the dialysis supply items 140 to thereby guide the user to interacting with the right object or control.

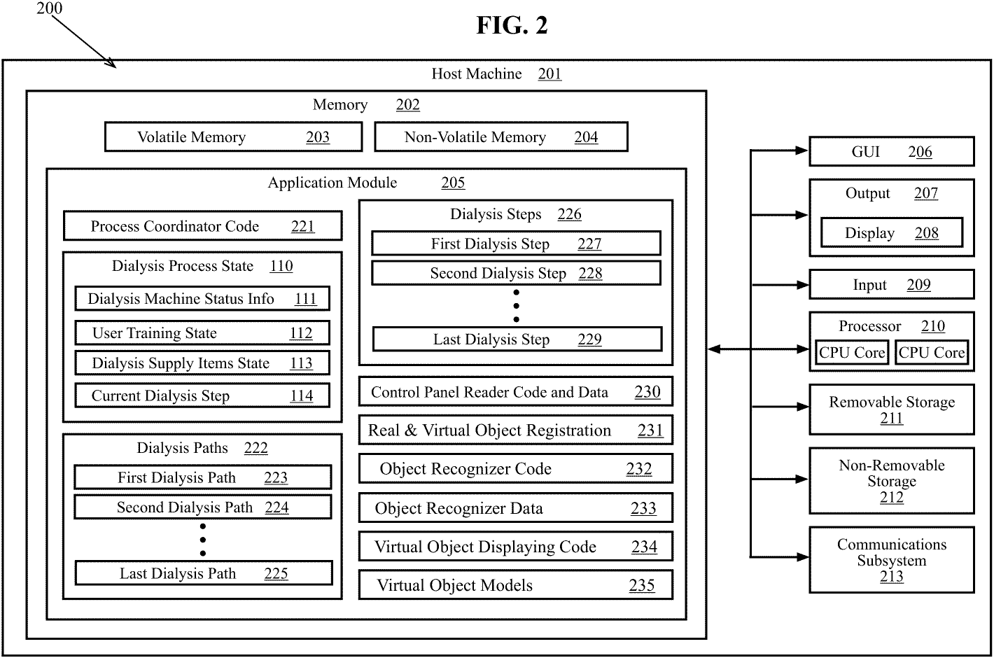

FIG. 2 is a high-level block diagram of a host machine that can provide guided dialysis training and supervision, according to some embodiments. A computing device in the form of a computer 201 configured to interface with controllers, peripheral devices, and other elements disclosed herein, may include one or more processing units 210, memory 202, removable storage 211, and non-removable storage 212. Memory 202 may include volatile memory 203 and non-volatile memory 204. Host machine 201 may include or have access to a computing environment that includes a variety of transitory and non-transitory computer-readable media such as volatile memory 203 and non-volatile memory 204, removable storage 211 and non-removable storage 212. Computer storage includes, for example, random access memory (RAM), read only memory (ROM), erasable programmable read-only memory (EPROM) and electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage, or other magnetic storage devices, or any other medium capable of storing computer-readable instructions as well as data. Of the listed computer storage, volatile memory and most RAM, such as dynamic RAM (DRAM), are transitory while the others are considered non-transitory.

Host machine 201 may include, or have access to, a computing environment that includes input 209, output 207, and a communications subsystem 213. The host machine 201 may operate in a networked environment using a communications subsystem 213 to connect to one or more remote computers, remote sensors and/or controllers, detection devices, hand-held devices, multi-function devices (MFDs), speakers, mobile devices, tablet devices, mobile phones, smartphone, or other such devices. The remote computer may also be a personal computer (PC), server, router, network PC, radio frequency identification (RFID) enabled device, a peer device or other common network node, or the like. The communication connection may include a local area network (LAN), a wide area network (WAN), Bluetooth connection, or other networks.

Output 207 is most commonly provided as a computer monitor or flat panel display but may include any output device. Output 207 and/or input 209 may include a data collection apparatus associated with host machine 201. In addition, input 209, which commonly includes a computer keyboard and/or pointing device such as a computer mouse, computer trackpad, touch screen, or the like, allows a user to select and instruct host machine 201. A user interface can be provided using output 207 and input 209. Output 207 may include a display 208 for displaying data and information for a user, or for interactively displaying a graphical user interface (GUI) 206. A GUI is typically responsive to user inputs entered through input 209 and typically displays images and data on display 208.

Note that the term “GUI” generally refers to a type of environment that represents programs, files, options, and so forth by means of graphically displayed icons, menus, and dialog boxes on a computer monitor screen or smart phone screen. A user can interact with the GUI to select and activate such options by directly touching the screen and/or pointing and clicking with a user input device 209 such as, for example, a pointing device such as a mouse, and/or with a keyboard. A particular item can function in the same manner to the user in all applications because the GUI provides standard software routines (e.g., the application module 205 can include program code in executable instructions, including such software routines) to handle these elements and report the user’s actions.

Computer-readable instructions, for example, program code in application module 205, can include or be representative of software routines, software subroutines, software objects, etc. described herein, are stored on a computer-readable medium and are executable by the processor device (also called a processing unit) 210 of host machine 201. The application module 205 can include computer code and data such as process coordinator code 221, dialysis process state 110, dialysis paths 222, dialysis steps 226, control panel reader code and data 230, real and virtual object registration 231, object recognizer code 232, object recognizer data 233, virtual object displaying code 234, virtual object models 235. The dialysis paths 222 can include a first dialysis path 223, a second dialysis path 224, and a last dialysis path 225. The dialysis steps 226 can include a first dialysis step 227, a second dialysis step 228, and a last dialysis step 229. For clarity, the dialysis path 101 illustrated in FIG. 1 includes dialysis steps. An equivalent implementation is for a dialysis path to include dialysis step indicators. An indicator can indicate one of the dialysis steps in dialysis steps 226. As such, the steps in a path can be reordered by changing the indicators and numerous dialysis paths can use the same dialysis step.

Control panel reader code and data 230 can be used to interpret the control panel of the dialysis machine and thereby produce the dialysis machine status information. Real and virtual object registration 231 can be data that indicates the locations and orientations of objects that are real or virtual. The object recognizer data 233 can include data that an algorithm can use to recognize an object in one or more images. The object recognizer code 232 can be computer code that, when executed, uses the object recognizer data 233 to recognize objects in images. As discussed above, object recognizer code is commercially and freely available and often comes with object recognizer data for common objects such as people, alphanumeric text, etc. Virtual object models 235 are data that describes how to display a virtual object such as a virtual avatar, a virtual dialysis machine, virtual dialysis supply items, etc. Virtual object displaying code 234 is computer code that when executed can use a virtual object model to display a virtual object to a user in the user’s virtual environment or augmented environment. A hard drive, CD-ROM, RAM, flash memory, and a USB drive are just some examples of articles including a computer-readable medium.

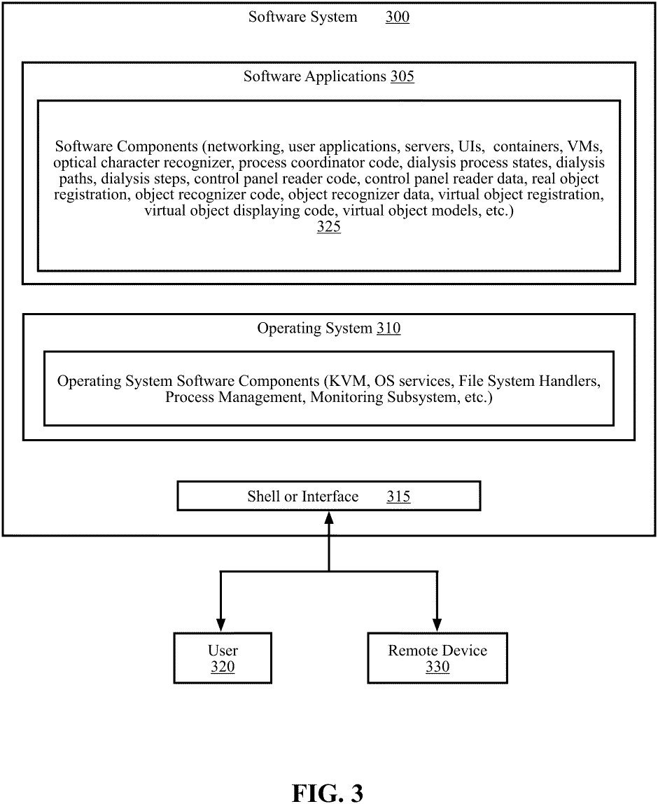

FIG. 3 is a high-level block diagram of a software system that can provide guided dialysis training and supervision, according to some embodiments. FIG. 3 illustrates a software system 300, which may be employed for directing the operation of data-processing systems such as host machine 201. Software applications 305, may be stored in memory 202, on removable storage 211 or on non-removable storage 212, and generally includes and/or is associated with an operating system 310 and a shell or interface 315. One or more application programs may be “loaded” (i.e., transferred from removable storage 211 or non-removable storage 212 into the memory 202) for execution by the host machine 201. Application programs 305 can include software components 325 such as software modules, software subroutines, software objects, network code, user application code, server code, UI code, container code, virtual machine (VM) code, optical character recognizer code, process coordinator code, dialysis process states, dialysis paths, dialysis steps, control panel reader code, control panel reader data, real object registration, object recognizer code, object recognizer data, virtual object registration, virtual object displaying code, virtual object models, etc. The software system 300 can have multiple software applications each containing software components. The host machine 201 can receive user commands and data through interface 315, which can include input 209, output 207, and communications connection 213 accessible by a user 320 or remote device 330. These inputs may then be acted upon by the host machine 201 in accordance with instructions from operating system 310 and/or software applications 305 and any software components 325 thereof.

Generally, software components 325 can include, but are not limited to, routines, subroutines, software applications, programs, modules, objects (used in object-oriented programs), executable instructions, data structures, etc., that perform particular tasks or implement particular abstract data types and instructions. Moreover, those skilled in the art will appreciate that elements of the disclosed methods and systems may be practiced with other computer system configurations such as, for example, hand-held devices, mobile phones, smartphones, tablet devices, multi-processor systems, microcontrollers, printers, copiers, fax machines, multi-function devices, data networks, microprocessor-based or programmable consumer electronics, networked personal computers, minicomputers, mainframe computers, servers, medical equipment, medical devices, and the like.

Note that the terms “component” and “module” as utilized herein may refer to one of or a collection of routines and data structures that perform a particular task or implement a particular abstract data type. Applications and components may be composed of two parts: an interface, which lists the constants, data types, variables, and routines that can be accessed by other modules or routines; and an implementation, which is typically private (accessible only from within the application or component) and which includes source code that actually implements the routines in the application or component. The terms application or component may also simply refer to an application such as a computer program designed to assist in the performance of a specific task such as word processing, accounting, inventory management. Components can be built or realized as special purpose hardware components designed to equivalently assist in the performance of a task.

The interface 315 can include a graphical user interface 206 that can display results, whereupon a user 320 or remote device 330 may supply additional inputs or terminate a particular session. In some embodiments, operating system 310 and GUI 206 can be implemented in the context of a “windows” system. It can be appreciated, of course, that other types of systems are possible. For example, rather than a traditional “windows” system, other operating systems such as, for example, a real time operating system (RTOS) more commonly employed in wireless systems may also be employed with respect to operating system 310 and interface 315. The software application 305 can include, for example, software components 325, which can include instructions for carrying out steps or logical operations such as those shown and described herein.

The description herein is presented with respect to embodiments that can be embodied in the context of, or require the use of, a data processing system such as host machine 201, in conjunction with program code in an application module 205 in memory 202, software system 300, or host machine 201. The disclosed embodiments, however, are not limited to any particular application or any particular environment. Instead, those skilled in the art will find that the system and method of the present invention may be advantageously applied to a variety of system and application software including database management systems, word processors, and the like. Moreover, the present invention may be embodied on a variety of different platforms including Windows, Macintosh, UNIX, Linux, Android, Arduino, and the like. Therefore, the descriptions of the exemplary embodiments, which follow, are for purposes of illustration and not considered a limitation.

Host machines 201 and software systems 300 can take the form of or run as virtual machines (VMs) or containers that run on physical machines. A VM or container typically supplies an operating environment, appearing to be an operating system, to program code in an application module and software applications 305 running in the VM or container. A single physical computer can run a collection of VMs and containers. In fact, an entire network data processing system including a multitude of host machines 201, LANs and perhaps even WANs or portions thereof can all be virtualized and running within a single computer (or a few computers) running VMs or containers. Those practiced in cloud computing are practiced in the use of VMs, containers, virtualized networks, and related technologies.

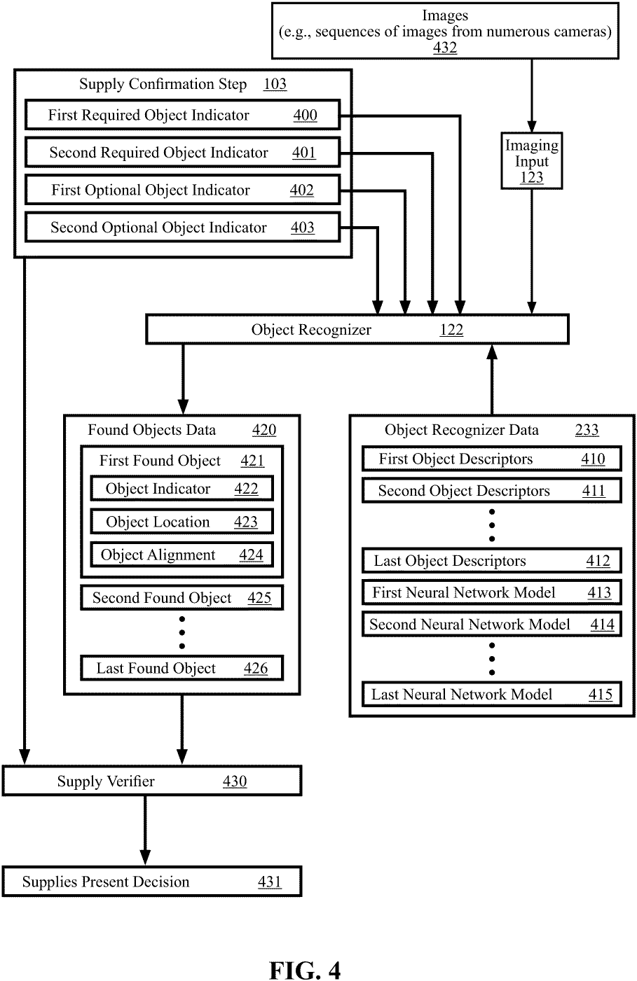

FIG. 4 is a high-level conceptual diagram of a supply confirmation step 103 being completed, according to aspects of the embodiments. The supply confirmation step 103 can include a first required object indicator 400, a second required object indicator 401, a first optional object indicator 402, and a second optional object indicator 403. The first required object indicator 400, the second required object indicator 401, the first optional object indicator 402, and the second optional object indicator 403 can each indicate a model or set of descriptors in the object recognizer data 233. The object recognizer data 233 can include first object descriptors 410, a second object descriptors 411, a last object descriptors 412, a first neural network model 413, a second neural network model 414, and a last neural network model 415. As is well known in the arts of image processing, objects can be recognized using descriptors or using neural networks. For example, a feature extraction program can be run on an image to thereby produce descriptors. The descriptors can be compared to descriptors for known objects to thereby determine if an object is present in the image, the location of the object, and the orientation of the object. It is also well known that a neural network can be trained to recognize objects in images. An image can be submitted to a trained neural network to thereby determine if an object is present in the image, the location of the object, and the orientation of the object.

The object recognizer 122 can receive images 432 via the imaging input 123. It is understood that there may be numerous cameras providing images to thereby image the patient setting from a variety of positions and angles. As is known in the art, images of an object that are obtained from numerous camera angles and positions may help refine calculations of the object’s location and orientation. The indicators in the supply confirmation step 103 indicate which descriptors and models the object recognizer 122 is to use for analyzing the images. The object recognizer 122 uses the descriptors and models to locate objects in the images. Based on the objects found, the object recognizer 122 produces found objects data 420 describing the found objects such as the first found object 421, the second found object 425, and the last found object 426. The data for a found object can include an object indicator 422, an object location 423, and an object orientation 424. The object indicator 422 identifies the object that was found. The object location 423 indicates where the object is located in the patient setting. The object orientation 424 indicates how the object is aligned within the patient setting. A supply verifier 430 uses the data in the supply confirmation step 103 and in the found objects data 420 to produce a supplies present decision 431 that indicates whether all of the required objects are present. The dialysis supply items state 113 can be updated to include the found objects, including their locations and orientations. The process coordinator 120 can move to the next step when supplies present decision 431 indicates all of the required objects are present.

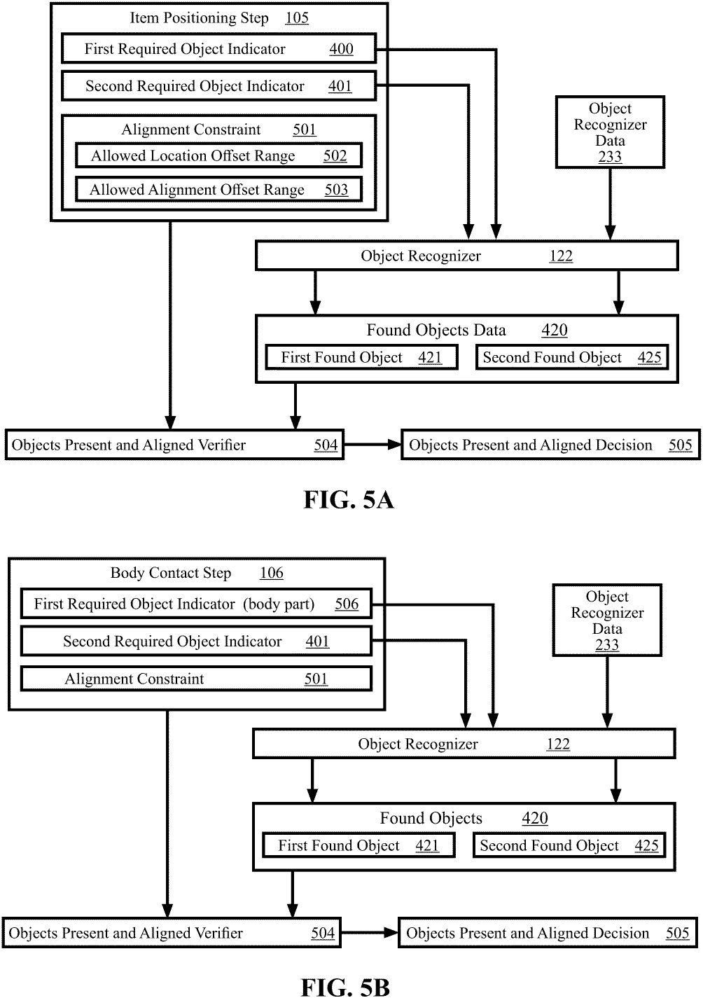

FIG. 5A is a high-level conceptual diagram of an item positioning step 105 being completed, according to aspects of the embodiments. The item positioning step 105 can include required object indicators and alignment constraints 501. The alignment constraints 501 can include an allowed location offset range 502 and an allowed alignment offset range 503. The allowed location offset range 502 indicates a range of allowed offsets between the objects. For example, the item positioning step may be for putting a dialysis bag in a fluid warmer. As such, one object is a dialysis bag and the other object is the fluid warmer. The dialysis bag is in the fluid warmer when the two objects are within the range of allowed offsets, thereby meeting the positioning criterion. The two objects are aligned relative to one another and the allowed alignment offset range 503 indicates the alignment range that is allowed. Returning to the example, the top of the dialysis may need to be positioned at the back of the fluid warmer. The allowed alignment offset range 503 can therefore be selected such that the dialysis bag and the fluid warmer are properly aligned. The discussion of FIG. 4 covered the aspects of locating objects, determining their alignment within the patient setting, and producing found objects data 420. The dialysis supply items state 113 can be updated to include the found objects, including their locations and orientations. An objects present and aligned verifier 504 can use a positioning criterion to make an objects aligned and present decision 505 based on the found objects data 420 or the dialysis supply items state 113. The positioning criterion may specify an allowed offset range (e.g., an offset range of [0, 40] can require that the objects are within 40 mm of one another). The process coordinator 120 can move to the next dialysis step when the required objects are present and aligned as indicated by the item positioning step 105.

FIG. 5B is a high-level conceptual diagram of a body contact step 106 being completed, according to aspects of the embodiments. An example of a body contact step is inserting a dialysis needle into an arm. The body contact step 106 is substantially the same as an item positioning step with the difference being that one of the objects is a body part of the patient. As such, the first required object indicator 506 may indicate descriptors or a neural net model that recognizes a body part.

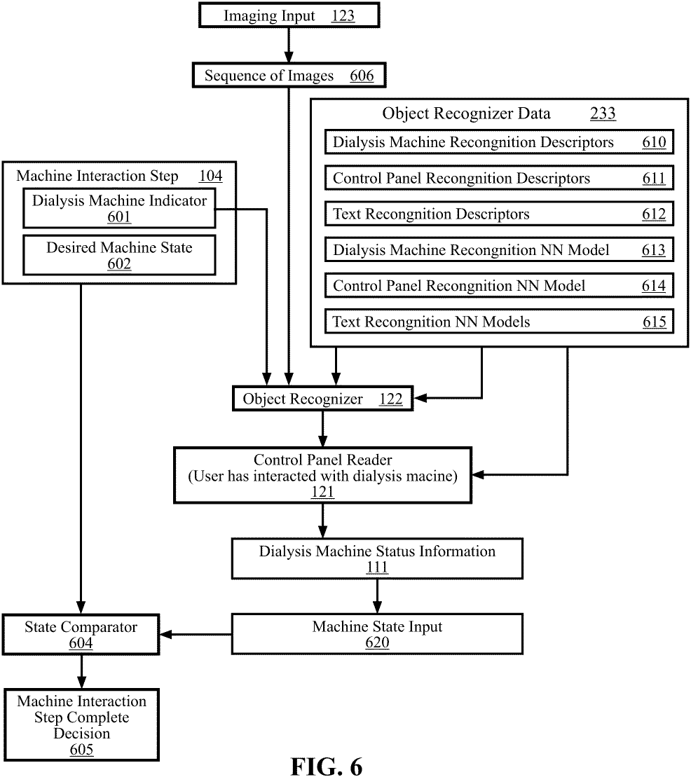

FIG. 6 is a high-level conceptual diagram of a machine interaction step 104 being completed, according to aspects of the embodiments. Object recognizer data 233 can be used for finding the dialysis machine within the patient setting, locating the control panel of the dialysis machine, and reading or interpreting the information on the control panel. As such, the object recognizer data 233 can include dialysis machine recognition data (e.g., dialysis machine recognition descriptors 610, dialysis machine recognition neural net model 613, etc.), control panel recognition data (e.g., control panel recognition descriptors 611, control panel recognition neural net model 614, etc.), and text recognition data (e.g., text recognition descriptors 612, text recognition neural net model 615, etc.). The machine interaction step 104 can include a dialysis machine indicator 601 and a desired machine state 602. The dialysis machine indicator 601 can indicate dialysis machine recognition data. The dialysis machine recognition data and the sequence of images can be used to find the dialysis machine in the patient setting. The control panel reader 121 can use the control panel recognition data and the text recognition data to read the control panel and produce dialysis machine status information 111 that may be written into the dialysis process state 110 and passed to a machine state input 620 that can provide the dialysis machine status information 111 to a state comparator 604. The state comparator 604 can compare the dialysis machine status information 111 to the desired machine state 602 and produce a machine interaction step complete decision 605 that indicates whether the machine interaction step 104 is complete. The process coordinator 120 can move to the next dialysis step when the dialysis machine status information 111 matches the desired machine state 602 as indicated by the machine interaction step 104. The machine state input is shown receiving the dialysis machine status information 111 from the control panel reader 121. An alternative is that the dialysis machine may have a wired or wireless input/output (I/O) port through which all or some of the dialysis machine status information 111 may be read. In such an alternative, data from the I/O port may be obtained and written into the dialysis process state 110 and passed to the state comparator 604.

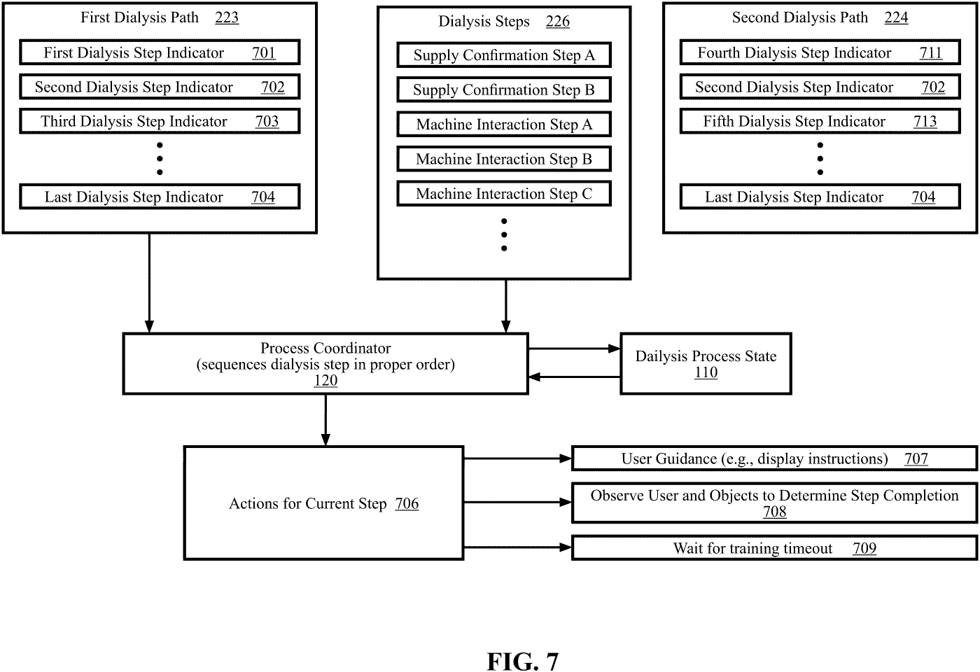

FIG. 7 is a high-level conceptual diagram of a process coordinator 120 using a first dialysis path 223 to guide a user through the proper order of dialysis steps of a dialysis procedure, according to aspects of the embodiments. The first dialysis path 223 and the second dialysis path 224 each include an ordered list of dialysis steps. Here, the dialysis steps are included in the dialysis paths by dialysis step indicators. Each dialysis step indicator indicates a dialysis step that is stored as one of the dialysis steps 226. The first dialysis path 223 includes a first dialysis step indicator 701, a second dialysis step indicator 702, a third dialysis step indicator 703, and a last dialysis step indicator 704. Performing the steps of the first dialysis path 223 in the proper order includes performing the step indicated by the first dialysis step indicator 701, then the step indicated by the second dialysis step indicator 702, then the step indicated by the third dialysis step indicator 703, and eventually the step indicated by the last dialysis step indicator 704. The second dialysis path 224 includes a fourth dialysis step indicator 711, the second dialysis step indicator 702, a fifth dialysis step indicator 713, and the last dialysis step indicator 704. Performing the steps of the second dialysis path 224 in the proper order includes performing the step indicated by the fourth dialysis step indicator 711, then the step indicated by the second dialysis step indicator 702, then the step indicated by the fifth dialysis step indicator 713, and eventually the step indicated by the last dialysis step indicator 704. As can be seen, the two dialysis paths include some of the same dialysis steps.

The process coordinator 120 is guiding the user through the steps of the first dialysis path 223. At each dialysis step, the process coordinator 120 performs, or calls on other programming to perform, the actions for the current step 706. The actions for the current step 706 can include providing user guidance 707 (e.g., displaying instructions), observing the user and objects to determine step completion 708, and waiting for a training timeout 709. Each step may include a training timeout for a timer that can be started at the start of the step. If the training timeout expires, then the user may receive additional guidance, the coach or monitor (e.g., a person assigned to the role) may intervene, etc.

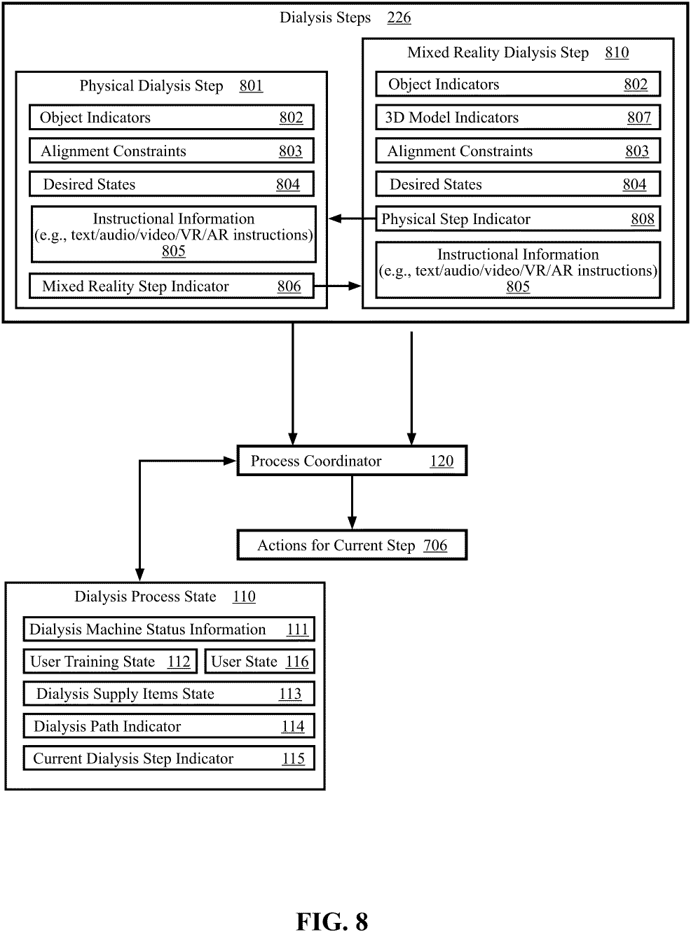

FIG. 8 is a high-level conceptual diagram of a process coordinator 120 using a user training state 112 to guide selection of a physical dialysis step 801 or a mixed reality dialysis step 810 as the current dialysis step, aspects of the embodiments. A physical dialysis step 801 can include object indicators 802, alignment constraints 803 (e.g., one or more positioning criterion), instructional information 805, and a mixed reality step indicator 806. The object indicators can indicate object recognition data (e.g., descriptors, neural net models, etc.) that can be used for recognizing a physical object in the patient setting. Alignment constraints 803 can be one or more alignment constraints 501 (also called a positioning criterion) for dialysis steps such as item positioning steps and body contact steps. Desired states 804 can indicate values or ranges for data items in the dialysis process state 110 that are required for completion of a dialysis step. Instructional information 805 is information that can be supplied to the user in order to guide the user to completing the step 801. The instructional information 805 can include information that is to be provided in the user’s augmented environment such as text overlying a physical object, data for displaying a virtual avatar to the user, audio that may be played or spoken by the virtual avatar, and information that is to be provided in some other manner. The mixed reality step indicator 806 can indicate a mixed reality dialysis step 810 that is similar to the physical dialysis step 801 with the exception that at least one of the objects is a virtual object (e.g., a virtual dialysis machine, a virtual tube, a virtual dialysis needle, etc.). The terms mixed reality, extended reality, and augmented reality are used interchangeably herein to refer to having an augmented environment in which virtual objects and virtual avatars are displayed to the user.

The mixed reality dialysis step 810 includes 3D model indicators 807 that can indicate a 3D model. The 3D model can be data that can be used by an output device to display a virtual object in the user’s augmented environment. Using 3D models to display virtual objects in augmented environments is well understood in the art. The output devices used for such presentations include virtual reality (VR) goggles, augmented reality (AR) goggles, projectors, and other devices. The mixed reality dialysis step 810 also includes a physical step indicator 808 that indicates the physical step 801. The process coordinator may move between the physical dialysis step 801 to the mixed reality dialysis step 810 based on the user training state 112. For example, an expired training timeout may set the user training state to indicate that the user should be shifted from the physical dialysis step 801 to the mixed reality dialysis step 810 in order to receive supplemental training. After completing the mixed reality dialysis step 810, the user may be moved back to the physical dialysis step 801.

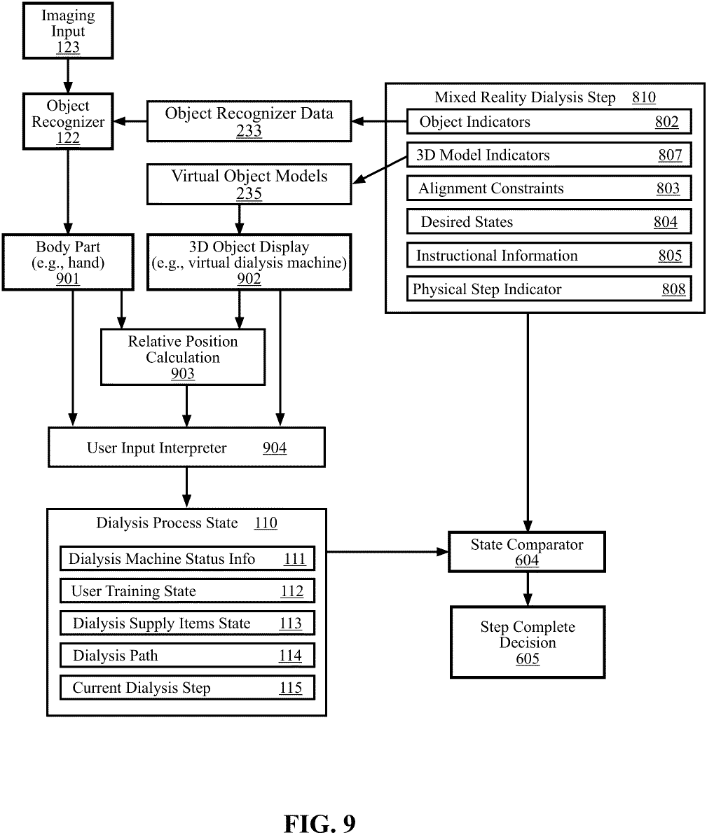

FIG. 9 is a high-level conceptual diagram of a mixed reality dialysis step 810 being completed, according to aspects of the embodiments. The object indicators 802 can indicate object descriptors or an object neural net model that the object recognizer 122 can use for recognizing a physical object in images received through an imaging input 123. The object descriptors or object neural net model can be stored as object recognizer data 233. The object recognizer 122 can determine the location and alignment of the objects, such as a body part 901 (e.g., the user’s hand), in the user’s physical environment. The user’s augmented environment can be the user’s physical environment augmented by virtual objects, which may include virtualized items (e.g., dialysis machines) and presentations of information. A floating text box near a physical object is a presentation of information in the user’s augmented environment. An avatar, such as a virtualized coach, pointing at objects (physical or virtual) or speaking to the user is also a presentation of information in the user’s augmented environment. The 3D model indicators 807 can indicate one or more of the virtual object models 235. The virtual object models 235 can be used to present virtual objects, such as 3D object display 902, to a user. When presented to the user, the virtual objects are located at specific positions and with specific alignments in the user’s augmented environment. Those specific positions and alignments may be obtained from the dialysis process state 110 or some other data structure. The user may interact with virtual objects to thereby change the virtual object’s position and alignment. The user may interact with virtual objects to thereby change the objects state. For example, moving the power switch to the “on” position can change the state of a virtual dialysis machine from “off” to “on”.

Relative position calculation 903 can receive the location and alignment of objects (real and virtual) and calculate the position of an object relative to another object. For example, the location and alignment of the user’s hand relative to the position and alignment of a virtual dialysis machine may be calculated for use in determining whether the user is interacting with the virtual dialysis machine. User input interpreter 904 can receive the relative position calculation 903 and determine the result of an interaction between the user and a virtual object. For example, the user may move an object such as a clamp or a dialysis machine control panel switch. The result of the interaction can be stored in the dialysis process state 110. A state comparator 604 can produce a step complete decision 605 when a desired state 804 is achieved.

Some machine interaction steps can involve interacting with a dialysis machine that is turned off. In FIG. 9, a virtual dialysis machine can be displayed to the user and the user’s movements relative to the virtual dialysis machine can result in changes to the dialysis process state. There is a point in the user’s training where a physical dialysis machine is introduced. The physical dialysis machine can be used for training without being powered on or without requiring an actual physical interaction. The object recognizer can recognize the physical dialysis machine. The location and alignment of the dialysis machine can be determined in the same manner that the location and alignment of any other object is determined. Thereafter, the user can interact with the physical dialysis machine in a manner similar to the interactions with a virtual dialysis machine. The relative position calculation 903 can receive the location and alignment of objects (real and virtual) and calculate the position of an object relative to another object. For example, the location and alignment of the user’s hand relative to the position and alignment of the physical dialysis machine may be calculated for use in determining whether the user is interacting with the physical dialysis machine. User input interpreter 904 can receive the relative position calculation 903 and determine the result of an interaction between the user and the physical dialysis machine. The result of the interaction can be stored in the dialysis process state 110. A state comparator 604 can produce a step complete decision 605 when a desired state 804 is achieved. As discussed above, user guidance can be displayed to the user in the user’s augmented environment and overlaying the physical dialysis machine. There are many advantages to training with a physical dialysis machine that is turned off or otherwise not fully operational. One advantage is that the training does not have to wait while the dialysis machine changes state in response to a user input. For example, a user can press a button (a machine interaction step) of a physical dialysis machine that causes the machine to perform a series of operations that may take many minutes to complete. The user may have to simply wait until the machine completes its operations. Such delays in the user’s training are not always necessary. By leaving the dialysis machine off, the user can press the button to thereby complete the step. The dialysis process state 110 may be updated as if the machine’s series of operations that are triggered by the button are completed. Another aspect of a dialysis machine that is turned off is that user guidance can be overlayed on top of the machine’s display or control panel. For example, a light can be made to appear illuminated on the control panel and a realistic display of information may be overlaid on the dialysis machine’s textual or graphical outputs. For example, a graphic, text, or images may by displayed overlaying a flat panel display such as a dialysis machine’s display panel. Some dialysis machines may need to be turned on in order to perform certain operations such as opening a panel that is locked shut by an electronic locking mechanism. Such interlocks may be used such that the machine cannot be opened during certain operational states. When the dialysis machine is turned off, the operation can be simulated and the result shown to the user in the user’s augmented environment. For example, a view of the opened panel and an interior view of the dialysis machine may be displayed.

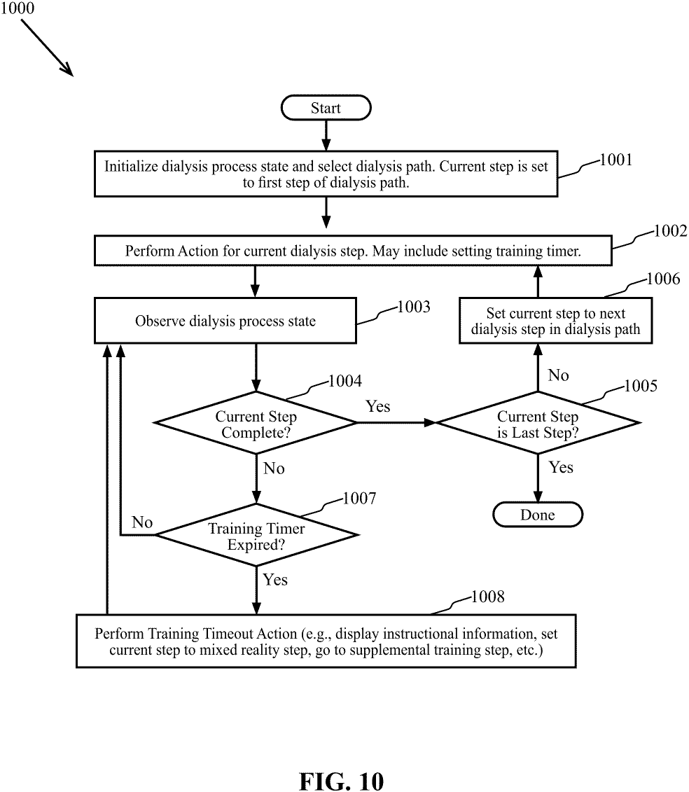

FIG. 10 is a high-level flow diagram of a process that a process coordinator may use to guide a user through a dialysis procedure, according to aspects of the embodiments. After the start, the process begins at block 1001. At block 1001, the process can initialize the dialysis process state, select a dialysis path, and set the current step to the first step of the dialysis path. At block 1002, the process can perform the action, which may include numerous operations, of the current dialysis step. The action may include setting a training timer. At block 1003, the process can observe the dialysis process state. At decision block 1004, the dialysis process state may be compared to a desired state of the current step to determine if the current step is complete. The process moves to decision block 1005 if the current step is complete at decision block 1004 and otherwise moves to decision block 1007. At decision block 1005, the process checks whether the current step is the last step in the dialysis path. The process is done if the current step is the last step in the dialysis path, otherwise the process moves to block 1006. At block 1006, the process sets the current step to the next dialysis step in the dialysis path before looping back to block 1002. At decision block 1007, the process can check whether the training timer has expired. The process can loop back to block 1003 if the training timer has not expired at decision block 1007, otherwise the process moves to block 1008. At block 1008 the process can perform the training timeout action for the current step before looping back to block 1003. Each dialysis step may include a training timeout action such as displaying instructional information, setting the current step to a mixed reality step, going to a supplemental training step, etc. The training timer is being used as a hint trigger. A hint trigger is an event that triggers the system to provide supplemental information to the user. The supplemental information that is provided to the user may appear at a hint location. The hint location is a location in the user’s augmented environment. For example, the hint location may be specified as overlying a specific dialysis supply item. In such a scenario, the location of that item may be obtained from the dialysis process state 110 and used as the hint location. Text or a marker may then be shown at the hint location in order to draw the user’s attention to the dialysis supply object.

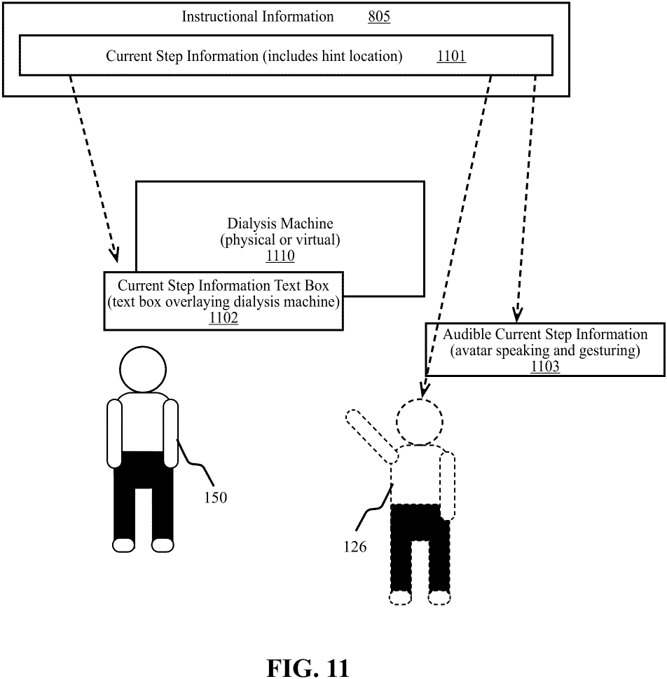

FIG. 11 is a high-level conceptual diagram of current step information 1101 being presented to a user, according to aspects of the embodiments. The instructional information 805 in a dialysis step can include current step information such as hinting at the location of a machine interaction, instructional audio, the positioning and content of a text overlay, the positioning and movement of an avatar, etc. For example, a current information text box 1102 may be displayed such that it overlays a dialysis machine 1110 (physical or virtual) in the user’s augmented environment. An avatar 126 of a virtualized coach or helper may point to the text box and appear to speak to the user 150. Audible current step information 1103 (audio recording, text-to-speech, etc.) may be played such that it appears that the avatar is providing voice instruction or may be played such that it seems to come from an invisible narrator (e.g., voice over).

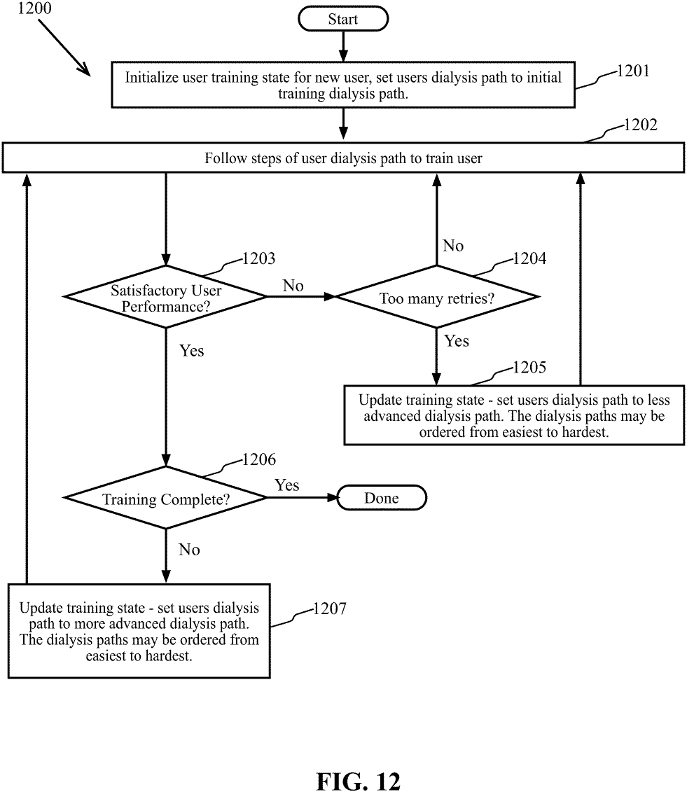

FIG. 12 is a high-level flow diagram of using a user training state to adjust the training of the user, according to aspects of the embodiments. After the start, the process begins at block 1201. At block 1201, the process can initialize the user training state for a new user and set the user dialysis path to an initial training dialysis path. At block 1202, the system and the user can follow the steps of the user dialysis path. At decision block 1203, the process can check whether the user’s performance is satisfactory. For example, satisfactory performance of a particular dialysis path may require that the user performed all the steps without supplemental coaching. The criteria for satisfactory performance may be stored in association with the dialysis paths. If the user’s performance is satisfactory at decision block 1203, the process moves to decision block 1206, otherwise the process moves to decision block 1204. At decision block 1204, the process can determine whether the user has attempted the current path too many times (e.g., number of tries exceeds a threshold value). The process can loop back to block 1202 if there has not been too many retries at decision block 1204, otherwise the process can move to block 1205. At block 1205, the process can update the user training state such that the user is presented with a different, easier, dialysis path. For example, the dialysis paths may be ordered from easiest to hardest and the user dialysis path may be set to the path that is just below the current user dialysis path in difficulty. The process can loop back to block 1202 from block 1205. At decision block 1206, the process can check whether training is complete. The process is done if the training is complete at decision block 1206, otherwise the process can move to block 1207. At block 1207, the training difficulty is increased before the process loops back to block 1202. For example, the user dialysis path may be set to the next most difficult path in an ordered set of dialysis paths. Note that a dialysis path used for training a user may be a path for a complete dialysis procedure or an incomplete dialysis procedure. A complete dialysis procedure is the full treatment that the user needs. An incomplete dialysis procedure may include some of the steps for a complete procedure, steps that use virtual dialysis supply items, steps that use a virtual dialysis machine, etc.

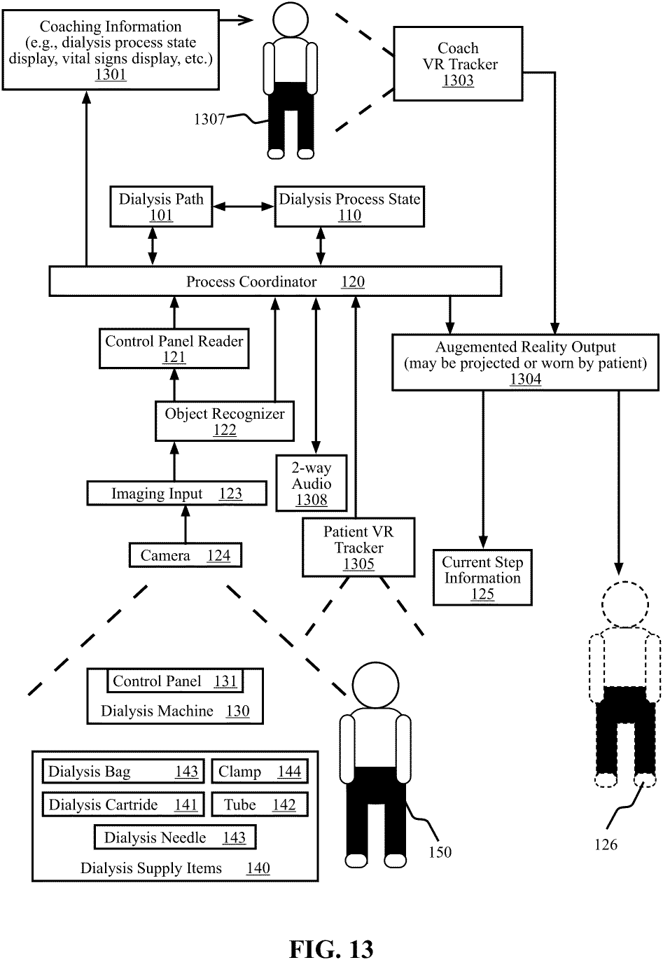

FIG. 13 is a high-level block diagram of a software system that can use a virtual avatar 126 to provide guided dialysis training and supervision, according to some embodiments. The process coordinator 120 is guiding the user 150 through the dialysis steps of a dialysis path 101. The user 150 has access to a dialysis machine 130 and dialysis supply items 140. A camera, object recognizer, and control panel reader 121 can be used to track the states of the dialysis machine and the dialysis supply items. The states can include the position and alignment of the dialysis machine 130, the dialysis supply items 140, etc. A patient VR tracker 1305 can track the location and alignment of the user 150 and the user’s body parts (hands, arms, legs, torso, etc.). The patient VR tracker can be a camera such as camera 124 or can be specialized body tracking hardware such as a Microsoft Kinect type device, Vive body tracking devices, etc. The process coordinator can use data structures such as the dialysis process state 110 to track the user, items, objects, and dialysis procedure.

The process coordinator 120 may detect that some level of intervention is needed. Intervention may be needed when a training timer expires, a dialysis supply item or other object disappears unexpectedly, a patient monitoring device obtains an out of bounds measurement, etc. In such cases, a coach 1307 may be alerted. The coach 1307 is a person who monitors patients (users) during dialysis procedures. Coaching information 1301 can be provided to the coach 1307. The coach VR tracker 1303, which may be similar to the patient VR tracker 1305, can provide positioning information to an augmented reality output 1304 that then shows an avatar 126 in the patient’s augmented environment. The movements of the coach 1307 can be replicated by the avatar to thereby provide instruction to the patient. The coach and the user 150 may communicate via a 2-way audio 1308 as is commonly done in current telepresence systems. The augmented reality output 1304 may also overlay textual and other information in the user’s augmented environment.

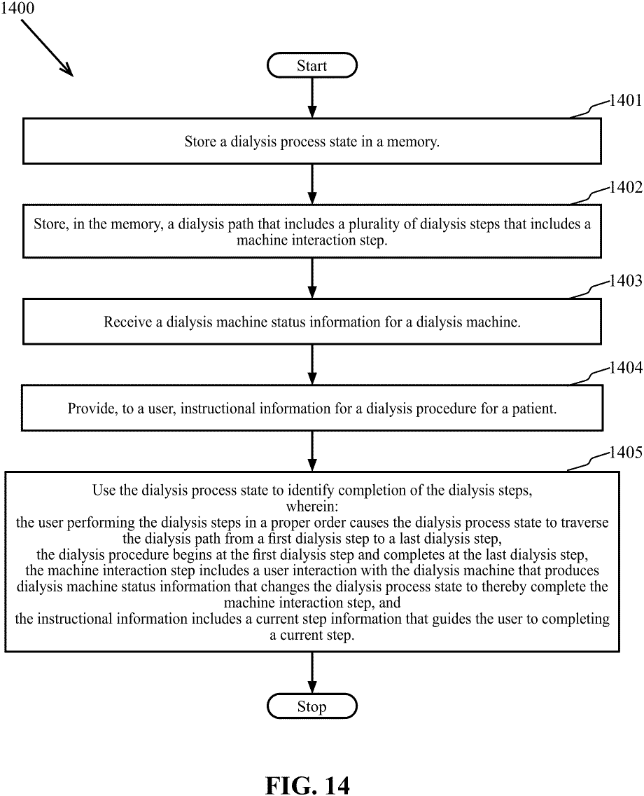

FIG. 14 is a high-level flow diagram illustrating a method for providing guided dialysis training and supervision, according to some embodiments. After the start, the method begins at block 1401. At block 1401 the method can store a dialysis process state in a memory. At block 1402 the method can store, in the memory, a dialysis path that includes a plurality of dialysis steps that includes a machine interaction step. At block 1403 the method can receive a dialysis machine status information for a dialysis machine. At block 1404 the method can provide, to a user, instructional information for a dialysis procedure for a patient. At block 1405 the method can use the dialysis process state to identify completion of the dialysis steps, wherein the user performing the dialysis steps in a proper order causes the dialysis process state to traverse the dialysis path from a first dialysis step to a last dialysis step, the dialysis procedure begins at the first dialysis step and completes at the last dialysis step, the machine interaction step includes a user interaction with the dialysis machine that produces dialysis machine status information that changes the dialysis process state to thereby complete the machine interaction step, and the instructional information includes a current step information that guides the user to completing a current step.

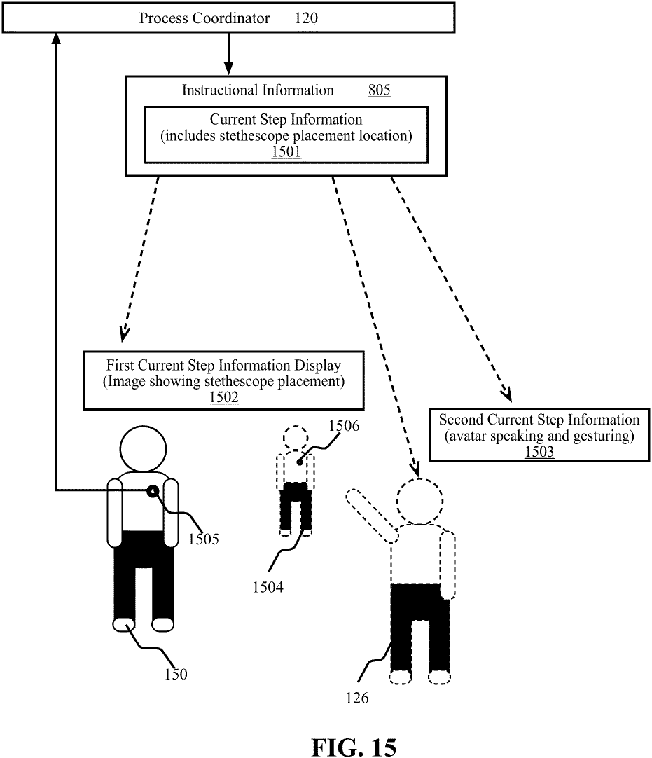

FIG. 15 is a high-level conceptual diagram of a virtualized avatar 126 guiding a patient 150 that is using a remotely readable stethoscope 1505, according to aspects of the embodiments. The process coordinator 120 is providing instructional information 805 to the user 150. The instructional information 805 includes current step information 1501 that instructs the user 150 to properly place the remotely readable stethoscope 1505 such that vital signs measurements can be returned. The current step information can include a first current step information display 1502 of a virtual figure 1504 on which a virtual stethoscope 1506 is positioned. A second current step information 1503 can include an avatar 126 that is speaking and gesturing toward the virtual figure 1504, the virtual stethoscope 1506, the patient 150, and the remotely readable stethoscope 1505 to thereby coach the patient in obtaining the vital signs measurement. The process coordinator may receive the vital signs measurement and ensure that it is within an allowable range. For example, a body contact step may include properly placing a remotely readable stethoscope at a specific location on the patient’s torso. Once the body contact step is complete, the process coordinator may obtain the vital signs measurement, compare it to an allowable range, and then select a subsequent step based on whether the vital signs measurement is in an allowable range or outside the allowable range. Another aspect is that the patient 150 may be monitored with cameras. As such, the virtual figure 1504 may be a rendering of the patient or an idealized rendering of the patient. An idealized rendering can be an image that omits details or certain body parts. An idealized image may accentuate certain details by, for example, changing the patient’s complexion, apparent body mass indicator, age, etc.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

While the above-described techniques are described in a general context, those skilled in the art will recognize that the above-described techniques may be implemented in software, hardware, firmware or any combination thereof. The above-described embodiments of the invention may also be implemented, for example, by operating a computer system to execute a sequence of machine-readable instructions. Typically, the computer readable instructions, when executed on one or more processors, implements a method. The instructions may reside in various types of computer readable media. In this respect, another aspect of the present invention concerns a programmed product, comprising a computer readable medium tangibly embodying a program of machine-readable instructions executable by a digital data processor to perform the method in accordance with an embodiment of the present invention. The computer readable media may comprise, for example, RAM (not shown) contained within the computer. Alternatively, the instructions may be contained in another computer readable media such as a magnetic data storage diskette and directly or indirectly accessed by a computer system. Whether contained in the computer system or elsewhere, the instructions may be stored on a variety of machine-readable storage media, such as a vital signs measurement conventional “hard drive”, a RAID array, magnetic tape, electronic read-only memory, an optical storage device (e.g., CD ROM, WORM, DVD, digital optical tape), paper “punch” cards. In an illustrative embodiment of the invention, the machine-readable instructions may comprise lines of compiled C, C++, or similar language code commonly used by those skilled in the programming.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the claims as described herein.

What is claimed is:

1. A system comprising:
a memory that stores a dialysis process state and a dialysis path that includes a plurality of dialysis steps that includes a machine interaction step;
a machine state input that receives dialysis machine status information for a dialysis machine;
an instruction output that provides instructional information for a dialysis procedure for a patient; and
a processor that uses the dialysis process state to identify completion of the dialysis steps, wherein:
a user performing the dialysis steps in a proper order causes the dialysis process state to traverse the dialysis path from a first dialysis step to a last dialysis step,
the dialysis procedure begins at the first dialysis step and completes at the last dialysis step,
the machine interaction step includes a user interaction with the dialysis machine that causes the dialysis machine status information to change the dialysis process state to thereby complete the machine interaction step,
the instructional information includes a current step information that guides the user to completing a current step, and
the instruction output provides the current step information to the user.

2. The system of claim 1, further including:
an imaging input, wherein:
the dialysis machine is a physical dialysis machine,
the imaging input receives a sequence of images of a control panel of the dialysis machine, and
the dialysis machine status information is determined using the images of the control panel.

3. The system of claim 1, wherein:
a user training state tracks a training level of the user;
the user training state is used to determine the instructional information that is presented to the user; and
the user training state is used to select a hint trigger that triggers display of the instructional information to the user.

4. The system of claim 1, wherein:
the dialysis machine is a virtual dialysis machine; and
the user interacts with the virtual dialysis machine to thereby change the dialysis machine status information.

5. The system of claim 1, wherein:
a 3D model of the dialysis machine is used to present the dialysis machine to the user in augmented reality, mixed reality, or extended reality.

6. The system of claim 1, wherein:
the instructional information is presented to the user in augmented reality, mixed reality, or extended reality;
the dialysis machine is a physical dialysis machine; and
a current dialysis step is used to determine a hint location at which the instructional information appears to the user.

7. The system of claim 1, further including:
an imaging input that receives a plurality of images; and
an object recognizer that recognizes a dialysis supply item in the images, wherein:
the dialysis steps include a supply confirmation step,
the dialysis supply item is imaged in the images,
the object recognizer uses the images to confirm that the dialysis supply item is present, and
the supply confirmation step is completed by confirming that the dialysis supply item is present.

8. The system of claim 1, further including:
an imaging input that receives a plurality of images; and
an object recognizer that recognizes a plurality of dialysis supply items in the images, wherein:
the dialysis supply items include a clamp, a tube, and a dialysis bag,
the dialysis steps include a supply confirmation step,
the dialysis supply items are imaged in the images,
the object recognizer uses the images to confirm that the dialysis supply items are present, and
the supply confirmation step is completed by confirming that the dialysis supply items are present.

9. The system of claim 1, further including:
an imaging input that receives a plurality of images; and
an object recognizer that recognizes a first dialysis supply item and a second dialysis supply item, wherein:
the dialysis steps include an item positioning step that includes confirming that the first dialysis supply item is properly positioned relative the second dialysis supply item,
the first dialysis supply item and the second dialysis supply item are imaged in the images,
the object recognizer uses the images to determine a first item position of the first dialysis supply item and a second item position of the second dialysis supply item, and
the item positioning step is completed by determining that the first item position relative to the second item position meets a positioning criterion.

10. The system of claim 9, wherein the first dialysis supply item is a tube and the second dialysis supply item is a clamp.

11. The system of claim 1, further including:
an imaging input that receives a plurality of images; and
an object recognizer that recognizes a body part of the patient and a dialysis supply item, wherein:
the dialysis steps include a body contact step that includes confirming that the dialysis supply item is properly positioned relative the body part,
the body part and the dialysis supply item are imaged in the images,
the object recognizer uses the images to determine an item position of the dialysis supply item and a body part position of the body part, and
the body contact step is completed by determining that the item position relative to the body part position meets a positioning criterion.

12. The system of claim 11, wherein the dialysis supply item is a dialysis needle.

13. The system of claim 1, wherein:
the current step information is provided to the user as an overlay that appears over the dialysis machine; and
the dialysis machine is a physical dialysis machine.

14. The system of claim 1, wherein the current step information is provided to the user by a virtual avatar that interacts with a virtual dialysis machine or virtual dialysis supply items.

15. A method comprising:
storing a dialysis process state in a memory;
storing, in the memory, a dialysis path that includes a plurality of dialysis steps that includes a machine interaction step;
receiving a dialysis machine status information for a dialysis machine;
providing, to a user, instructional information for a dialysis procedure for a patient; and
using the dialysis process state to identify completion of the dialysis steps, wherein:
the user performing the dialysis steps in a proper order causes the dialysis process state to traverse the dialysis path from a first dialysis step to a last dialysis step,
the dialysis procedure begins at the first dialysis step and completes at the last dialysis step,
the machine interaction step includes a user interaction with the dialysis machine that produces dialysis machine status information that changes the dialysis process state to thereby complete the machine interaction step, and
the instructional information includes a current step information that guides the user to completing a current step.

16. The method of claim 15, further including:
receiving a sequence of images of a control panel of the dialysis machine; and
using the images of the control panel to determine the dialysis machine status information,
wherein the dialysis machine is a physical dialysis machine.

17. The method of claim 15, wherein:
a user training state tracks a training level of the user;
the user training state is used to determine the instructional information that is presented to the user; and
the user training state is used to select a hint trigger that triggers display of the instructional information to the user.

18. The method of claim 15, wherein:
the current step information is provided to the user as an overlay that appears over the dialysis machine; and
the dialysis machine is a physical dialysis machine.

19. The method of claim 15, wherein the current step information is provided to the user by a virtual avatar that interacts with a virtual dialysis machine or virtual dialysis supply items.

20. A system comprising:
a means for storing a dialysis process state and a dialysis path that includes a plurality of dialysis steps that includes a step for machine interaction;
a means for using a dialysis machine status information for a dialysis machine to change the dialysis process state;
an instructive means for instructing a user for performing a dialysis procedure for a patient; and
a means identifying completion of the dialysis steps using dialysis process state, wherein:
the user performing the dialysis steps in a proper order causes the dialysis process state to traverse the dialysis path from a first dialysis step to a last dialysis step,
the dialysis procedure begins at the first dialysis step and completes at the last dialysis step,
the step for machine interaction produces dialysis machine status information that changes the dialysis process state to thereby complete the step for machine interaction, and
the instructive means includes a means for guiding the user to complete a current step.
